(19) United States
(12) Patent Application Publication
(10) Pub. No.: US 2015/0007823 A1
(43) Pub. Date: Jan. 8, 2015

(54) METHOD FOR CALIBRATING THE LEVEL OF OXYGEN IN RESPIRATORY GAS RELATED TO THE LEVEL OF OXYGEN IN THE BLOOD CIRCULATION OF AN INDIVIDUAL

(71) Applicant: Mermaid Care A/S, Nørresundby (DK)

(72) Inventors: Stephen Edward Rees, Aalborg (DK); Dan Stieper Karbing, Aalborg (DK); Lars Pilegaard Thomsen, Aalborg (DK)

(21) Appl. No.: 14/382,278

(22) PCT Filed: Mar. 1, 2013
(86) PCT No.: PCT/DK2013/050052

§ 371 Date: Aug. 29, 2014

(30) Foreign Application Priority Data
Mar. 2, 2012 (DK) PA 2012 00171

(51) Int. Cl. A61M 16/10; A61B 5/1455; A61B 5/00; A61M 16/00

(52) U.S. Cl. CPC A61M 16/1005; A61M 16/0051; A61B 5/14551; A61B 5/4836; A61M 2016/1025; A61M 2230/205; A61M 2230/50; A61M 2230/005; A61M 2205/3303
USPC 128/204.23

(57) ABSTRACT

The present invention relates to a method for calibrating, or adjusting, the level of oxygen in respiratory gas related to the level of oxygen in the blood circulation of the individual, comprising providing a level of oxygen in the gas flow passing into, or out, of the respiratory system of the individual and producing a corresponding first output, providing a level of oxygen in the blood circulation of the individual and producing a corresponding second output, providing a computer for receiving and storing at least two measurements, each measurement being the concurrent output of said first output and said second output within a data structure, in which the two stored outputs are mutually related, in data storage means associated with the computer, the at least two measurements being conducted at respective levels of oxygen in the gas flow passing into the respiratory system, and calibrating the level of oxygen in the gas value in response to a delay between the level of oxygen in the blood circulation of the individual and the level of oxygen in the gas flow passing into, or out, of the respiratory system of the individual.

### TECHNICAL FIELD OF THE INVENTION

The present invention relates to a method for calibrating the level of oxygen in respiratory gas related to the level of oxygen in the blood circulation of an individual, and a corresponding computer program product.

### BACKGROUND OF THE INVENTION

In clinical practice, measurement of pulmonary gas exchange is limited to simple single parameter measurements such as the PaO2/FiO2 ratio or effective pulmonary shunt which have been criticized for providing insufficient descriptive detail (Karbing et al. 2007). In contrast, detailed experimental methods have been applied to improve physiological understanding, for instance the multiple inert gas elimination technique (MIGET) (Wagner, Saltzman & West 1974), but these methods have not found their way into routine clinical practice. Recently, a new method based on measurements of arterial oxygenation at varied inspired oxygen fractions and a physiological mathematical model, has been developed. The method known as ALPE (acronym for Automatic Lung Parameter Estimator system) provides the clinician with estimates of pulmonary shunt, and ventilation perfusion mismatch (Rees et al. 2002), which can be used to determine optimal inspired oxygen fraction (Karbing et al. 2010). To describe a patient's gas exchange ALPE conducts a 3 to 5 step maneuver taking 10 to 15 minutes, where inspired oxygen (FiO2) is changed and the resulting end-tidal (FetO2) and pulse oximetry arterial oxygenation (SpO2) measured. FIG. 1 illustrates an example of data collected during an ALPE maneuver, with raw data shown as points and the drawn curve is the best fit of the mathematical model of pulmonary gas exchange included in ALPE (Karbing et al. 2011).

Clinical studies have evaluated the use of ALPE in post-operative hypoxaemia (Rasmussen et al. 2006, Rasmussen et al. 2007, Kjaergaard et al. 2001, Kjaergaard et al. 2004), to describe a range of intensive care patients (Karbing et al. 2007, Kjaergaard et al. 2003) and in patients with left-sided heart failure (Moesgaard et al. 2009). The model has been compared to MIGET (Rees et al. 2006, Rees et al. 2010).

The mathematical model included in ALPE describes transport of oxygen from inspired gas to the blood and includes conservation of mass equations at steady state. This dictates that measurements of arterial oxygenation and FetO₂ must be at steady state conditions following a change in FiO₂ level. During an ALPE maneuver, steady-state is evaluated by monitoring changes in breath by breath FetO₂ where a continuous low variation (plateau) over a period of time is defined as a steady state, typically achieved within 2 to 4 minutes.

The assumption of steady state may be insufficient in some cases: 1) In patients with severe lung disease, for example chronic obstructive pulmonary disease (COPD), 2 to 4 minutes may be insufficient to reach steady state. Indeed several studies have shown that this can take as long as 30 minutes (Woolf 1976, Sherter et al. 1975); 2) The ALPE system evaluates steady state by a constant FetO₂ value, however arterial oxygenation may have a slower time course, taking longer to equilibrate than FetO₂; and 3) The ALPE system approximates arterial oxygen saturation using pulse oximetry measurement of SpO₂ at the fingertip which is delayed in relation to arterial values due to circulation time of blood and sensor averaging (Young et al. 1992, Zubieta-Calleja et al. 2005). Circulation can for example be compromised at the pulse oximeter site due to well-known mechanisms such as vasoconstriction. The delay is patient specific as it depends on local blood flow in the fingers (Ding et al. 1992) and is affected by hypothermia (MacLeod et al. 2005).

Hence, improved methods for obtaining respiratory parameters relating to an individual would be advantageous.

### SUMMARY OF THE INVENTION

Since a steady state may not be achieved in 2 to 4 minutes in all individuals, a method is presented inhere wherein breath by breath measurements of oxygen parameters such as FetO2 and SpO2 are determined to e.g. rapidly estimate one or more respiratory parameters relating to an individual. Thus, the need for steady state levels of oxygen levels in the blood are eliminated, which otherwise slows the method. This method is based on that the delay due to e.g. pulse oximetry can be calibrated for, on an individual patient basis.

Thus, an object of the present invention relates to a method for calibrating the level of oxygen in respiratory gas related to the level of oxygen in the blood circulation of an individual.

Thus, one aspect of the invention relates to a method for calibrating the level of oxygen in respiratory gas related to the level of oxygen in the blood circulation of the individual, comprising
providing a level of oxygen in the gas flow passing into, or out, of the respiratory system of the individual and producing a corresponding first output,
providing a level of oxygen in the blood circulation of the individual and producing a corresponding second output,
providing a computer for receiving and storing at least two measurements, each measurement being the concurrent output of said first output and said second output within a data structure, in which the two stored outputs are mutually related, in data storage means associated with the computer, the at least two measurements being conducted at respective levels of oxygen in the gas flow passing into the respiratory system, and
calibrating the level of oxygen in the gas value in response to a delay between the level of oxygen in the blood circulation of the individual and the level of oxygen in the gas flow passing into, or out, of the respiratory system of the individual.

The invention is particularly, but not exclusively, advantageous for obtaining an improved method which provides significantly reliable and/or faster measurements of respiratory parameters of patients as compared to previous methods applied, such as the so-called ALPE. For example measurements performed on patients suffering from chronic obstructive pulmonary disease (COPD) may be performed an order of magnitude quicker and also more reliable because the required assumption of steady state may be uncertain and/or difficult to validate.

In the context of the present invention, the concept of ‘calibrating the level of oxygen in the gas value’ is to be understood in the broadest sense. Thus, the term ‘calibrating’ may alternatively be replaced, or having synonyms meaning, with adjusting, compensating, regulating, bringing into line, correcting, adapting, and so forth, as the skilled person will understand once the general teaching and principle of the present invention has been appreciated, in particular when realizing the origin and meaning of the delay between the level of oxygen in the blood circulation of the individual and the level of oxygen in the gas flow passing into, or out, of the respiratory system of the individual. Notice that the delay as such may be caused by a number of factors, the physiological delay typically being the dominating source without being limited to this particular type of delay.

In one embodiment of the invention, the provided values of the level of oxygen in the blood circulation of the individual (e.g. SpO2) are shifted in time (either positive or negative depending on the reference) according to the delay. In another embodiment, the provided values of the level of respiratory oxygen of the individual (e.g. FEO2) are shifted in time (either positive or negative depending on the reference) according to the delay.

In one alternative embodiment of the invention, the actual values of the level of blood circulation of the individual (e.g. SpO2) and/or the level of respiratory oxygen of the individual (e.g. FEO2) are calibrated, or adjusted, according to the delay in order to provide faster and/or more reliable measurement of respiratory parameters. This could be done by using an appropriate physiological model as the skilled person will appreciate, this could be performed as an alternative or addition to the said shifting in time.

In one embodiment, wherein the computer may be further adapted for estimating least two respiratory parameters relating to the individual, the two respiratory parameters descriptive of the pulmonary gas exchanges. The computer may specifically be adapted for determining at least two respiratory parameters chosen from the list consisting of: Rdiff, shunt, \dot{V}/\dot{Q}, \dot{V}-distribution, \dot{Q}-distribution, H-shift, V-shift, or CO2-shift, or any combination thereof, or equivalents or derived parameters thereof as the skilled person will appreciate once the general principle and teaching of the present invention has been fully comprehended.

In particular, the present invention may be implemented in connection with a device for determining respiratory parameters as described in U.S. Pat. No. 7,008,380 (assigned to Mermaid Care A/S), which is hereby incorporated by reference in its entirety. Cf. also Rees et al., 2002. In a separate aspect, the present invention can accordingly be implemented in a device for determining one or more respiratory parameters, particularly at least two parameters, relating to an individual, comprising

a gas flow device having means for conducting a flow of inspiratory gas from an inlet opening to the respiratory system of the individual and a flow of expiratory gas from the respiratory system of the individual to an outlet opening,

a gas-mixing unit for supplying a substantially homogeneous gas to the inlet opening of the gas flow device,

first supply means for supplying a first gas to an inlet of the gas mixing unit and having first control means for controlling the flow of the first gas,

second supply means for supplying a second gas having an oxygen fraction different to the gas supplied from the first supply means to an inlet of the gas mixing unit and having second control means for controlling the flow of the second gas,

a computer for determining said one or more respiratory parameters,

first detection means for detecting the level of oxygen (SaO2, SpO2, PaO2, PpO2) in the blood circulation of the individual and producing an output to the computer accordingly, and

second detection means for detecting the level of oxygen (FIO2, FE'O2, FĒ O2, PIO2, PE'02, PĒO2) in the gas flow passing into or out of the respiratory system of the individual and producing an output to the computer accordingly, the computer being adapted for retrieving and storing at least two measurements being the concurrent output produced by the first detection means and the second detection means within a data structure, in which the two stored outputs are mutually related, in data storage means associated with the computer, the at least two measurements being conducted at respective levels of oxygen in the gas flow passing into the respiratory system, the computer further being adapted for determining at least one respiratory parameter (Rdiff, shunt, \dot{V}/\dot{Q}, H-shift, V-shift) being descriptive of the condition of the individual, the determination being based on the at least two measurements.

Additionally, or alternatively, the present invention may be implemented in connection with another device for determining respiratory parameters as described in international patent application WO 2012/069051 (assigned to Mermaid Care A/S), which is also hereby incorporated by reference in its entirety. The latter device may estimate pulmonary parameters indicative of gas exchange of both O2 and CO2 in an individual. Thus, the present invention may be implemented in a device for determining at least two respiratory parameters relating to an individual, comprising

a gas flow device having means for conducting a flow of inspiratory gas from an inlet opening to the respiratory system of the individual and a flow of expiratory gas from the respiratory system of the individual to an outlet opening,

a gas-mixing unit for supplying a substantially homogeneous gas to the inlet opening of the gas flow device,

first supply means for supplying a first gas to an inlet of the gas mixing unit and having first control means for controlling the flow of the first gas,

second supply means for supplying a second gas having an oxygen fraction different to the gas supplied from the first supply means to an inlet of the gas mixing unit and having second control means for controlling the flow of the second gas,

a computer for determining said two or more respiratory parameters,

first detection means for detecting the level of oxygen in the blood circulation of the individual and producing an output to the computer accordingly, and

second detection means for detecting the level of oxygen in the gas flow passing into or out of the respiratory system of the individual and producing an output to the computer,

first carbon dioxide detection means for detecting the level of carbon dioxide in the blood circulation of the individual and producing an output to the computer accordingly, and

second carbon dioxide detection means for detecting the level of carbon dioxide in the gas flow passing into or out of the respiratory system of the individual and producing an output to the computer accordingly,

the computer being adapted for retrieving and storing at least two oxygen measurements and one carbon dioxide measurement,

the oxygen measurements being the concurrent output produced by the first detection means and the second detection means within a data structure, in which the two stored outputs are mutually related and related to a stored oxygen measurement at a corresponding level of oxygen in the gas flow passing into the respiratory system,

the carbon dioxide measurement being the concurrent output produced by the first carbon dioxide detection means and the second carbon dioxide detection means within a data structure, in which the two stored outputs are mutually related and related to a stored carbon dioxide measurement at a corresponding level of oxygen in the gas flow passing into the respiratory system, and

the computer further being adapted for determining at least two respiratory parameters being descriptive of the pulmonary gas exchange of oxygen and/or carbon dioxide of the individual, the determination being based on the at least two oxygen measurements and one carbon dioxide measurement.

Both of the above-mentioned devices may beneficially exploit that the calibration the level of oxygen in the gas value in response to a delay between the level of oxygen in the blood circulation of the individual and the level of oxygen in the gas flow passing into, or out, of the respiratory system of the individual, may result in much improved measurement time and/or reliability.

Another aspect of the present invention relates a computer program product comprising software code adapted to enable the computer to calibrating the level of oxygen in respiratory gas related to the level of oxygen in the blood circulation of the individual according to the preceding aspect. This aspect of the invention is particularly, but not exclusively, advantageous in that the present invention may be accomplished by a computer program product enabling a computer system to carry out the operations of the apparatus/system of the first aspect of the invention when down- or uploaded into the computer system. Such a computer program product may be provided on any kind of computer readable medium, or through a network.

The individual aspects of the present invention may each be combined with any of the other aspects. These and other aspects of the invention will be apparent from the following description with reference to the described embodiments.

### BRIEF DESCRIPTION OF THE FIGURES

The method according to the invention will now be described in more detail with regard to the accompanying figures. The figures show one way of implementing the present invention and is not to be construed as being limiting to other possible embodiments falling within the scope of the attached claim set.

FIG. 1

FIG. 1 shows an ALPE model representation, curve, of end-tidal oxygen fraction (FetO2) versus pulse oximetry arterial oxygenation (SpO2) measured at steady state for different inspired oxygen fractions (FiO2). Circle, FiO2=21%; square, FiO2=18%; triangle, FiO2=15%; tipped triangle FiO2=30%.

FIG. 2

FIG. 2 shows pulse oximetry arterial oxygenation (SpO2), solid line, left axis, and inspired oxygen fraction (FiO2), dashed line, right axis, plotted against time. The label A marks the first change in FiO2. The grey vertical dashed line marked B shows the beginning of response of SpO2. Duration from A to B is the estimate of delay in SpO2.

FIG. 3

FIG. 3 shows breath-by-breath FetO2 (or FEO2) versus SpO2 raw data (top) and calibrated for SpO2 delay (bottom) for a representative patient included in the study (grey). Steady state oxygen levels for each inspired oxygen fraction FiO2) step are marked in black by: Circle=21%, Square=18%, Triangle=15%, Diamond=25%, Flipped triangel=30%, Pentagram=35%.

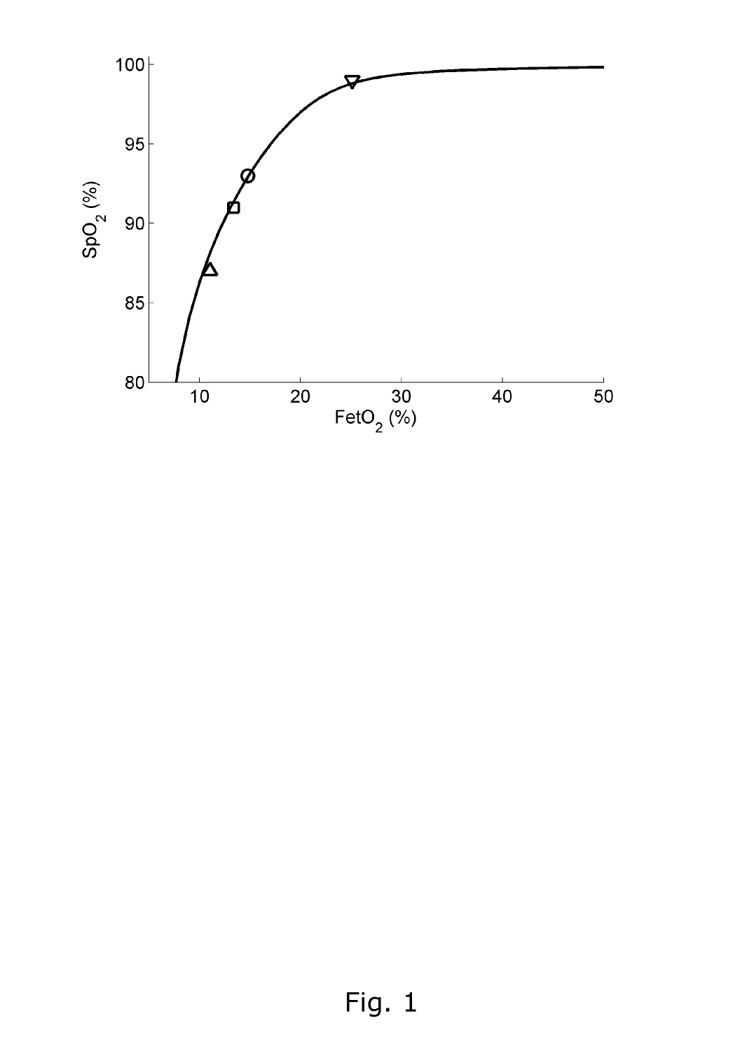

The present invention will now be described in more detail in the following.

### DETAILED DESCRIPTION OF THE INVENTION

The invention relates a method for calibrating the level of oxygen in respiratory gas related to the level of oxygen in the blood circulation of the individual.

The method comprises providing a level of oxygen in the gas flow passing into, or out, of the respiratory system of the individual, such as FEO2, and producing a corresponding first output.

The method further comprises providing a level of oxygen in the blood circulation of the individual, such as SpO2, and producing a corresponding second output,

A computer is provided for receiving and storing the at least two measurements, each measurement being the concurrent output of said first output and said second output within a data structure, in which the two stored outputs are mutually related, in data storage means associated with the computer, the at least two measurements being conducted at respective levels of oxygen in the gas flow passing into the respiratory system. Further, the invention comprises the step of calibrating, or adjusting, the level of oxygen in the gas value in response to a delay between the level of oxygen in the blood circulation of the individual and the level of oxygen in the gas flow passing into, or out, of the respiratory system of the individual.

Advantageously, the level of oxygen in respiratory gas may be provided by measurements of FiO₂, PiO₂, FE'O₂, FE'O2, PE'O₂ and/or PEO₂, or other equivalent measures available to the skilled person.

Advantageously, the level of oxygen in the blood circulation of the individual is provided by measurements of SaO₂, CaO₂, PaO₂, SpO₂, and/or PpO₂, or other equivalent measures available to the skilled person.

Typically, wherein the level of oxygen is in the gas flow passing into the respiratory system may be different in the at least two measurements. In a particular embodiment, the level of oxygen in at least one measurement may be the level natural present in air measured at sea level, such as around 21%.

The delay may be measured, estimated or fitted, possibly a combination of these ways of finding the delay may be applied.

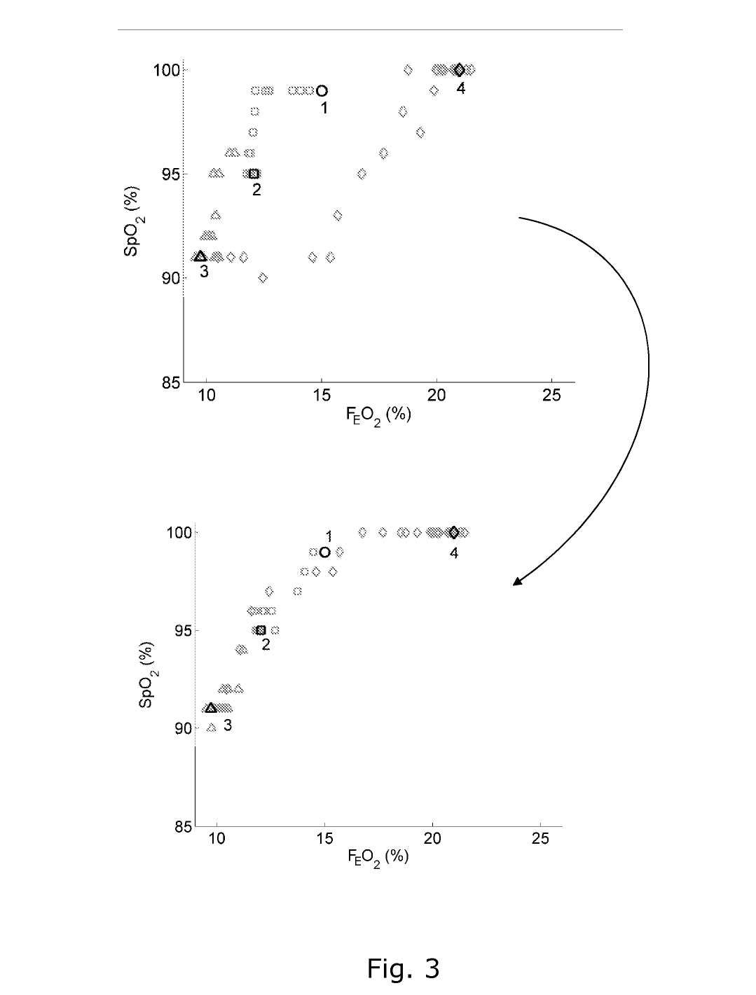

In one embodiment, the provided values of the level of oxygen in the blood circulation of the individual (SpO2) are shifted in time according to the delay, e.g. for the delay of 28 seconds for patient number 1, the patient data for oxygen in the blood being depicted in FIG. 3 top are shifted by 28 seconds in the bottom of FIG. 3.

Alternatively, the provided values of the level of respiratory oxygen of the individual (FEO2) may be shifted in time according to the delay, corresponding to negative shift of 28 seconds for patient number, this is however not shown in FIG. 3, but the result would have been the same.

Alternatively or additionally, the delay may be estimated for a specific patient condition, such as sex, age, diagnosis, disease history, weight, a local perfusion level, a temperature change, and/or an average based on a patient group.

Alternatively or additionally, the delay may be obtained by a fitting by minimizing deviation from a predefined curve, such as a function or a polynomial, and/or a physiological/mathematical model of oxygen transport. Appropriate minimization techniques are ready available to the skilled person once the general teaching of the present invention is comprehended. As seen in FIG. 3 (bottom), the calibrated values can be fitted to a relatively smooth or continuous function.

Typically, the at least two measurements may be provided from measurements obtained at different time points. The different time points being preferably shifted in time by at least one breath of the individual from which the measurements have been obtained, optionally two breaths, three breaths, fours breaths, etc. In particular, the at least two measurements at different time points may be obtained at non-steady state conditions for the respiratory state of the individual yielding faster measurement time. The measurements may be obtained after inhaling or exhaling. Advantageously, further measurements may also be shifted in time by at least one breath of the subject from which the data points have been obtained, e.g. 3 measurements, 4 measurements, 5 measurements, 6 measurements, etc.

Advantageously, the time between the first measurement and the second measurement is less than 2 minutes, such as less than 1 minute such as less than 30 seconds. The present invention is particularly advantageous in that an equilibrium or steady state condition is not necessary.

The present invention may be beneficially applied when the individual is a normal person, or suffers from one or more respiratory diseases or abnormalities, including primary and secondary lung diseases, such as chronic obstructive pulmonary disease (COPD), acute lung injury, acute respiratory distress syndrome, pulmonary edema, asthma, pleural disease, or airway disease. Other related or similar diseases/conditions for which the present invention may be advantageously applied are also contemplated.

In a particular aspect, the invention relates to a computer program product comprising software code adapted to enable the computer to calibrating the level of oxygen in respiratory gas related to the level of oxygen in the blood circulation of the individual according to any of preceding claims. Thus, advantageously the invention may be implemented on a computer having appropriate software and relevant patient data stored in connection with the computer

The invention can be implemented by means of hardware, software, firmware or any combination of these. The invention or some of the features thereof can also be implemented as software running on one or more data processors and/or digital signal processors.

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLE 1

Pulse oximetry SpO₂ delay was evaluated and individual patient calibration explored in 16 patients diagnosed with COPD.

### Patients

Sixteen patients admitted to the local pulmonary out-patient clinic all suffering from mild to very severe COPD entered in this study. Patients, 31% women, where 68±11 years and had a FEV₁% of 56±24. All patients gave their informed consent and the study was approved by the local Ethics Committee. An ALPE manoeuvre was performed for each patient (Mermaid Care A/S, DK9400 Nørresundby, Denmark). During the investigation the equipment continuously records FetO2, and SpO2.

### Data Analysis

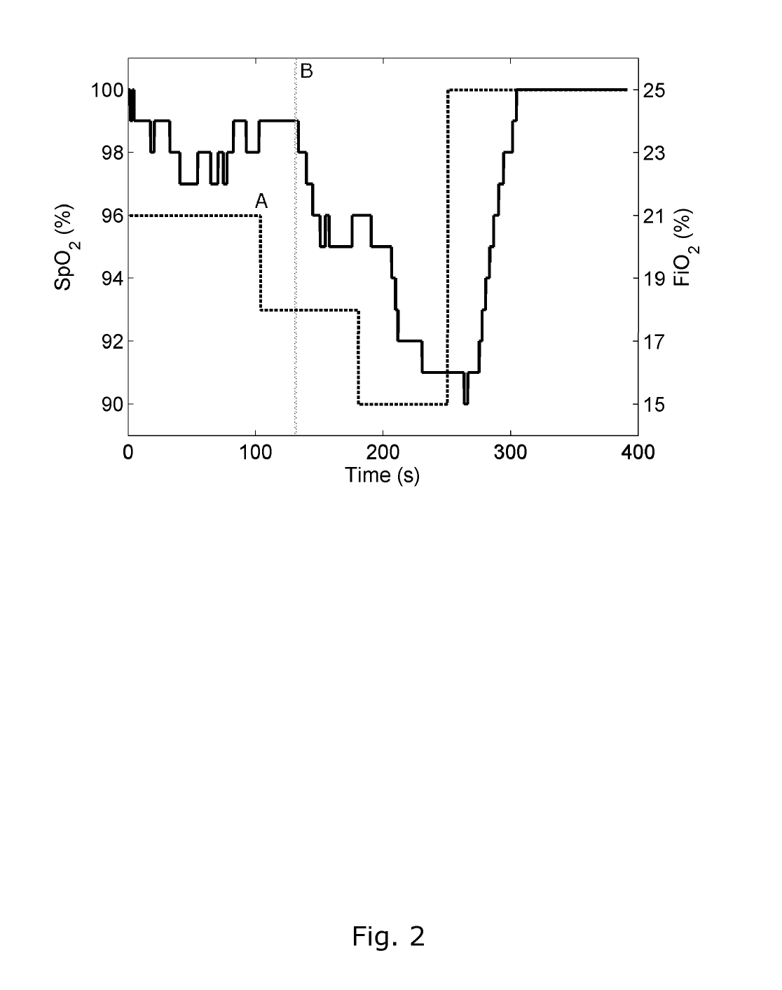

For each patient delay in SpO2 is estimated using a plot of SpO2 and FiO2 over time for the entirety of the ALPE experiment (FIG. 2). Delay is estimated as the duration from the first change in FiO2 (A) to the beginning of response of SpO2 (B, dashed vertical line). To perform this calibration is was necessary that a sufficiently large signal was seen in SpO2 so as to determine the start of SpO2 change from noise. In 8 of the 16 patients this was not the case for the first step change in FiO2 and a subsequent step was therefore used.

To explore whether FetO2 versus SpO2 data lie on a single continuous curve and to examine the effect of SpO2 delay, two plots were then made for each patient, with FetO2 plotted against SpO2 for both raw data and with SpO2 data corrected for delay. Expirations shorter than one second were excluded in these plots to prevent error in measurements of FetO2 due to lack of alveolar plateau.

### Results

Table 1 gives values for SpO2 delay for all patients. The average delay was 39.6 seconds, a value which is within range of the 30 seconds reported for finger probe pulse oximeters (MacLeod et al. 2005) where hyperthermia was absent.

| Patient no | Delay |
|---|---|
| 1 | 28 |
| 2 | 23 |
| 3 | 38 |
| 4 | 38 |
| 5 | 41 |
| 6 | 42 |
| 7 | 31 |
| 8 | 29 |
| 9 | 37 |
| 10 | 47 |
| 11 | 45 |
| 12 | 40 |
| 13 | 37 |
| 14 | 40 |
| 15 | 55 |
| 16 | 70 |
| Average | 39.6 |
| Standard deviation | 11.2 |
| Minimum | 23 |
| Maximum | 70 |

FIG. 3 shows FetO2 versus SpO2 raw data (top) and calibrated for SpO2 delay (bottom) for one patient included in the study, the lower graph being derived from the upper graph as indicated by the arrow connecting the upper and lower graph. Typical profiles of FetO2 versus SpO2 change during FiO2 steps can be seen in patient 1 both for calibrated and un-calibrated data. The start points for oxygen steps are marked with black symbols and are numbered to illustrate the order of steps. For patient 1, the ALPE experiment starts at a FiO2=21% (black circle), followed by a reduction in FiO2 to 18%. For the un-calibrated data in this patient, immediately following the change in FiO2, SpO2 remains constant while FetO2 reduces. After approximately 6 breaths SpO2 reduces with a relatively constant FetO2. The first step change in FiO2 ends at the data point marked with a black square. The subsequent reduction FiO2 from 18% to 15% has a similar profile with an initial drop in FetO2 followed by a reduction in SpO2. In the last step FiO2 is increased from 15% to 25%. Once again FetO2 changes occur first followed by change in SpO2. The errors are such that on reducing FiO2 data is shifted up and to the left, and on increasing FiO2 data is shifted down and to the right. Similar patterns are seen in all patients. Following calibration or adjustment, data lie on a single continuous curve including both breath by breath data and the final ALPE equilibrium points, illustrating that difference in dynamics between FetO2 and SpO2 could be explained by the SpO2 delay.

### Conclusion

In this example it is shown that data describing breath by breath FetO2 and SpO2 corrected for delay lie on a single curve. This illustrates that, even in patients with COPD, FetO2 and SaO2 may have similar time constants and complete oxygen equilibration can be avoided in order to draw an FetO2 versus SpO2 curve suitable for estimating pulmonary gas exchange.

COPD patients were studied here as these reflect those expected to have greater duration of oxygen equilibration than normal. Despite this selection the patients studied here had FEV1% level in the more moderate range of COPD and only four could be classified as severe COPD according to the GOLD criteria.
Thus, this example study has shown that a lack of equilibrium between FetO2 and SpO2 may not be a limitation when step changes in FiO2 and mathematical models are used as a tool for estimating pulmonary gas exchange.

### Glossary

FiO2 Fraction of oxygen in inspired gas.
PiO2 Pressure of oxygen in inspired gas.
SaO2 Oxygen saturation of arterial blood, measured from a blood sample.
CaO2 Oxygen concentration in arterial blood.
PaO2 Pressure of oxygen in arterial blood, measured from a blood sample.
SpO2 Oxygen saturation of arterial blood, measured transcutaneously.
PpO2 Pressure of oxygen in arterial blood, measured transcutaneously.
FE'O2 Fraction of oxygen in expired gas at the end of expiration.
FEO2 Fraction of oxygen in the mixed expired gas.
PE'O2 Pressure of oxygen in expired gas at the end of expiration.
PEO2 Pressure of oxygen in the mixed expired gas.

Non-limiting list of respiratory parameters descriptive of the pulmonary gas exchange:
shunt Respiratory parameter representing the fraction of blood not involved in gas exchange.
Rdiff Respiratory parameter representing a resistance to oxygen diffusion across the alveolar lung capillary membrane.
\dot{V} Ventilation.
\dot{Q} Perfusion.
\dot{V}/\dot{Q} Respiratory parameter representing the balance between ventilation and perfusion of a homogeneous region of the lung.
\dot{V}-distribution Respiratory parameter representing fraction of ventilation going to different regions of the lungs or fraction of ventilation going to different ventilated compartments of a model of pulmonary gas exchange.
\dot{V}-distribution Respiratory parameter representing fraction of perfusion going to different regions of the lungs or fraction of perfusion going to different ventilated compartments of a model of pulmonary gas exchange.
V-shift Respiratory parameter representing a vertical shift in plots of FiO2 against SaO2 , FiO2 against SpO2, FE'O2 against SaO2, or FE'O2 against SpO2.
H-shift Respiratory parameter representing a horizontal shift in plots of FiO2 against SaO2 , FiO2 against SpO2, FE'O2 against SaO2, or FE'O2 against SpO2.
CO2-shift Respiratory parameter representing the CO2-level shift in plots of FiCO2 against PaCO2 , FiCO2 against PtcCO2, FE'CO2 against PaCO2, or FE'CO2 against PtcCO2.

In short, the present invention relates to a method for calibrating, or adjusting, the level of oxygen in respiratory gas related to the level of oxygen in the blood circulation of the individual, comprising providing a level of oxygen in the gas flow passing into, or out, of the respiratory system of the individual and producing a corresponding first output, providing a level of oxygen in the blood circulation of the individual and producing a corresponding second output, providing a computer for receiving and storing at least two measurements, each measurement being the concurrent output of said first output and said second output within a data structure, in which the two stored outputs are mutually related, in data storage means associated with the computer, the at least two measurements being conducted at respective levels of oxygen in the gas flow passing into the respiratory system, and calibrating the level of oxygen in the gas value in response to a delay between the level of oxygen in the blood circulation of the individual and the level of oxygen in the gas flow passing into, or out, of the respiratory system of the individual.

### REFERENCES

Ding, Z. N., Shibata, K., Yamamoto, K., Kobayashi, T. & Murakami, S. 1992, “Decreased circulation time in the upper limb reduces the lag time of the finger pulse oximeter response”, Canadian journal of anaesthesia=Journal canadien d'anesthesie, vol. 39, no. 1, pp. 87-89.
Karbing, D. S., Allerod, C., Thorgaard, P., Carius, A. M., Frilev, L., Andreassen, S., Kjaergaard, S. & Rees, S. E. 2010, “Prospective evaluation of a decision support system for setting inspired oxygen in intensive care patients”, Journal of critical care, vol. 25, no. 3, pp. 367-374.
Karbing, D. S., Kjaergaard, S., Andreassen, S., Espersen, K. & Rees, S. E. 2011, “Minimal model quantification of pulmonary gas exchange in intensive care patients”, Medical engineering & physics, vol. 33, no. 2, pp. 240-248.
Karbing, D. S., Kjaergaard, S., Smith, B. W., Espersen, K., Allerod, C., Andreassen, S. & Rees, S. E. 2007, “Variation in the PaO2/FiO2 ratio with FiO2: mathematical and experimental description, and clinical relevance”, Critical care (London, England), vol. 11, no. 6, pp. R118.
Kjaergaard, S., Rees, S., Malczynski, J., Nielsen, J. A., Thorgaard, P., Toft, E. & Andreassen, S. 2003, “Non-invasive estimation of shunt and ventilation-perfusion mismatch”, Intensive care medicine, vol. 29, no. 5, pp. 727-734.
Kjaergaard, S., Rees, S. E., Gronlund, J., Nielsen, E. M., Lambert, P., Thorgaard, P., Toft, E. & Andreassen, S. 2004, “Hypoxaemia after cardiac surgery: clinical application of a model of pulmonary gas exchange”, European journal of anaesthesiology, vol. 21, no. 4, pp. 296-301.
Kjaergaard, S., Rees, S. E., Nielsen, J. A., Freundlich, M., Thorgaard, P. & Andreassen, S. 2001, “Modelling of hypoxaemia after gynaecological laparotomy”, Acta Anaesthesiologica Scandinavica, vol. 45, no. 3, pp. 349-356.
MacLeod, D. B., Cortinez, L. I., Keifer, J. C., Cameron, D., Wright, D. R., White, W. D., Moretti, E. W., Radulescu, L. R. & Somma, J. 2005, “The desaturation response time of finger pulse oximeters during mild hypothermia”, Anaesthesia, vol. 60, no. 1, pp. 65-71.
Moesgaard, J., Kristensen, J. H., Malczynski, J., Hoist-Hansen, C., Rees, S. E., Murley, D., Andreassen, S., Frokjaer, J. B. & Toft, E. 2009, “Can new pulmonary gas exchange parameters contribute to evaluation of pulmonary congestion in left-sided heart failure?”, The Canadian journal of cardiology, vol. 25, no. 3, pp. 149-155.
Rasmussen, B. S., Laugesen, H., Sollid, J., Gronlund, J., Rees, S. E., Toft, E., Gjedsted, J., Dethlefsen, C. & Tonnesen, E. 2007, “Oxygenation and release of inflammatory mediators after off-pump compared with after on-pump coronary artery bypass surgery”, Acta Anaesthesiologica Scandinavica, vol. 51, no. 9, pp. 1202-1210.
Rasmussen, B. S., Sollid, J., Rees, S. E., Kjaergaard, S., Murley, D. & Toft, E. 2006, “Oxygenation within the first 120 h following coronary artery bypass grafting. Influence of systemic hypothermia (32 degrees C.) or normothermia (36 degrees C.) during the cardiopulmonary bypass: a randomized clinical trial”, Acta Anaesthesiologica Scandinavica, vol. 50, no. 1, pp. 64-71.
Rees, S. E., Kjaergaard, S., Andreassen, S. & Hedenstierna, G. 2010, “Reproduction of inert gas and oxygenation data: a comparison of the MIGET and a simple model of pulmonary gas exchange”, Intensive care medicine, vol. 36, no. 12, pp. 2117-2124.
Rees, S. E., Kjaergaard, S., Andreassen, S. & Hedenstierna, G. 2006, “Reproduction of MIGET retention and excretion data using a simple mathematical model of gas exchange in lung damage caused by oleic acid infusion”, Journal of applied physiology (Bethesda, Md.: 1985), vol. 101, no. 3, pp. 826-832.
Rees, S. E., Kjaergaard, S., Thorgaard, P., Malczynski, J., Toft, E. & Andreassen, S. 2002, “The automatic lung parameter estimator (ALPE) system: non-invasive estimation of pulmonary gas exchange parameters in 10-15 minutes”, Journal of clinical monitoring and computing, vol. 17, no. 1, pp. 43-52.
Sherter, C. B., Jabbour, S. M., Kovnat, D. M. & Snider, G. L. 1975, “Prolonged rate of decay of arterial PO2 following oxygen breathing in chronic airways obstruction”, Chest, vol. 67, no. 3, pp. 259-261.
Wagner, P. D., Saltzman, H. A. & West, J. B. 1974, “Measurement of continuous distributions of ventilation-perfusion ratios: theory”, Journal of applied physiology, vol. 36, no. 5, pp. 588-599.
Woolf, C. R. 1976, “Letter: Arterial blood gas levels after oxygen therapy”, Chest, vol. 69, no. 6, pp. 808-809.
Young, D., Jewkes, C., Spittal, M., Blogg, C., Weissman, J. & Gradwell, D. 1992, “Response time of pulse oximeters assessed using acute decompression”, Anesthesia and Analgesia, vol. 74, no. 2, pp. 189-195.
Zubieta-Calleja, G. R., Zubieta-Castillo, G., Paulev, P. E. & Zubieta-Calleja, L. 2005, “Non-invasive measurement of circulation time using pulse oximetry during breath holding in chronic hypoxia”, Journal of physiology and pharmacology: an official journal of the Polish Physiological Society, vol. 56 Suppl 4, pp. 251-256.

1. A method for calibrating the level of oxygen in respiratory gas related to the level of oxygen in the blood circulation of an individual, comprising:
providing a level of oxygen in the gas flow passing into, or out, of the respiratory system of the individual and producing a corresponding first output,
providing a level of oxygen in the blood circulation of the individual and producing a corresponding second output,
providing a computer for receiving and storing at least two measurements, each measurement being the concurrent output of said first output and said second output within a data structure, in which the two stored outputs are mutually related, in a data storage means associated with the computer, the at least two measurements being conducted at respective levels of oxygen in the gas flow passing into the respiratory system, and
calibrating the level of oxygen in the gas value in response to a delay between the level of oxygen in the blood circulation of the individual and the level of oxygen in the gas flow passing into, or out, of the respiratory system of the individual,
wherein the delay is measured, estimated or fitted, and the provided values of the level of respiratory oxygen of the individual (FEO2) are shifted in time according to the delay.

2-20. (canceled)

21. The method according to claim 1, wherein the level of oxygen in respiratory gas is provided by measurements of FiO₂, PiO₂, FE'O₂, FE'O2, PE'O₂ and/or PEO₂.

22. The method according to claim 1, wherein the level of oxygen in the blood circulation of the individual is provided by measurements of SaO₂, CaO₂, PaO₂, SpO₂, and/or PpO₂.

23. The method according to claim 1, wherein the level of oxygen in the gas flow passing into the respiratory system is different in the at least two measurements.

24. The method according to claim 23, wherein the level of oxygen in at least one measurement is the level naturally present in air measured at sea level.

25. The method according to claim 1, wherein the provided values of the level of oxygen in the blood circulation of the individual (SpO2) are alternatively shifted in time according to the delay.

26. The method according to claim 1, wherein the delay is estimated for sex, age, diagnosis, disease history, weight, a local perfusion level, a temperature change, and/or an average based on a patient group.

27. The method according to claim 1, wherein the delay is obtained by a fitting procedure by minimizing deviation from a predefined curve.

28. The method according to claim 1, wherein the at least two measurements are provided from measurements obtained at different time points.

29. The method according claim 28, wherein the at least two measurements at different time points are obtained at non-steady state conditions for the respiratory state of the individual.

30. The method according to claim 28, wherein the different time points are shifted in time by at least one breath of the individual from which the measurements have been obtained.

31. The method according to claim 30, wherein the measurements are obtained after inhaling or exhaling.

32. The method according to claim 28, wherein further measurements are also shifted in time by at least one breath of the subject from which the data points have been obtained.

33. The method according to claim 1, wherein the time between a first measurement and a second measurement of the at least two measurements is less than 2 minutes.

34. The method according to claim 1, wherein the computer is further adapted for estimating at least two respiratory parameters relating to the individual, the at least two respiratory parameters being descriptive of the pulmonary gas exchange.

35. The method according to claim 34, wherein the computer is adapted for determining at least two respiratory parameters chosen from: Rdiff, shunt, \dot{V}/\dot{Q}, \dot{V}-distribution, \dot{Q}-distribution, H-shift, V-shift, or CO2-shift, or any combination thereof, or equivalents or derived parameters thereof.

36. The method according to claim 1, wherein the individual is a normal person, or a person that suffers from one or more respiratory diseases or primary or secondary lung diseases.

37. A computer program product comprising software code adapted to enable the computer to calibrating the level of oxygen in respiratory gas related to the level of oxygen in the blood circulation of the individual according to claim 1.
